(19) United States
(12) Patent Application Publication
(10) Pub. No.: US 2020/0384013 A1
(43) Pub. Date: Dec. 10, 2020

(54) COMPOSITION FOR IMPROVEMENT OF SYMPTOMS OF SPINAL CORD INJURY

(71) Applicant: MiZ Company Limited, Kanagawa (JP)

(72) Inventors: Hirokazu Kawaoka, Kanagawa (JP); Yusuke Ichikawa, Kanagawa (JP); Fumitake Satoh, Kanagawa (JP)

(21) Appl. No.: 16/896,843

(22) Filed: Jun. 9, 2020

(30) Foreign Application Priority Data
Jun. 10, 2019 (JP) 2019-108027

(51) Int. Cl. A61K 33/00; A61K 9/00

(52) U.S. Cl. CPC A61K 33/00; A61K 9/0043

(57) ABSTRACT

The present application provides a composition for improving a symptom of spine/spinal cord injury in a subject, the composition comprising molecular hydrogen as an active ingredient; and a method for improving a symptom of spine/spinal cord injury in a subject, the method comprising administering the composition to the subject.

### CROSS-REFERENCE TO RELATED APPLICATION

The present application claims priority to Japanese Patent Application No. 2019-108027 (filed on Jun. 10, 2019), the disclosure of which is incorporated herein in its entirety.

### FIELD OF THE INVENTION

The present invention provides a composition for improving a symptom of spine/spinal cord injury (or simply “spinal cord injury”).

The present invention also relates to a method for improving a symptom of spine/spinal cord injury in a subject having spine/spinal cord injury, the method comprising administering the composition to the subject.

### DESCRIPTION OF THE RELATED ART

Spine/spinal cord injury generally refers to a pathological condition in which the spinal column has been subjected to a strong external force, thus damaging the spine, and injuring the spinal cord. Such injury tends to be caused mainly by an accident such as a traffic accident, a fall, a downfall or a plunge. The injury involves disorders such as complete spinal cord injury, incomplete spinal cord injury, Brown-Sequard syndrome and complications depending on the site of injury, the degree (level) of dysfunction or the like. These disorders will be briefly described below. The complete spinal cord injury causes flaccid paralysis, loss of every sense and reflex activity, autonomic nervous system dysfunction below the level of injury, and the like, which are immediate and complete (J. E. Wilberger et al., Spine/Spinal Cord Trauma-22. Trauma and Intoxication-MSD Manual Professional Version; https://www.msdmanuals.com/ja-jp). The incomplete spinal cord injury causes incomplete loss of motions and senses, which is either permanent or temporary. The Brown-Sequard syndrome results from hemisection of the spinal cord, and causes ipsilateral spastic paralysis, loss of position sense and loss of contralateral pain and temperature senses. The complications include thrombosis, urinary tract infection, pneumonia, spasm, autonomic hyperreflexia, pain and burning sensation. Further, in the spinal cord injury, the injury is augmented through inflammatory immune response, accumulation of free radicals and apoptosis by, for example, release of an excess neurotransmitter and release of cytokine from injured cells.

Since the spinal cord is made up of the central nervous system, injury of the spinal cord may cause complete or incomplete loss of the motor function or the sensory function. The spinal cord is normally protected by the spine, and when the spine is injured by a traffic accident, an accidental downfall or the like as described above, injury of the spinal cord often occurs. If the nerve ruptures, the lower body is paralyzed because the nerve is not regenerated. In recent years, an attempt has been made to treat spinal cord injury by regeneration medicine using cells such as neural progenitor cells and mesenchymal stem cells.

In addition, spinal cord injury model experiment results have been reported which show that hydrogen is effective for protection of nerves subjected to spinal cord injury (X. Chen et al., Cell Physiol. Biochem. 2018; 47: 176-190 and C. Chen et al., Neurochem. Res. 2010; 35(7): 1111-1118). Molecular hydrogen is known to have a function of suppressing oxidative stress by neutralizing active oxygen species. These documents suggest that by administering high-concentration hydrogen of, for example, 50% or more to a model of injured nerve cells of the spinal cord extracted from a rat or a mouse, or a spinal cord-injured animal model, oxidative stress was suppressed, and as a result, the nerve cells of the spinal cord were protected. However, there are no evident findings for improvement of a symptom (e.g. aftereffects) of spine/spinal cord injury by hydrogen in vivo in animals such as humans or dogs which have been subjected to spinal cord injury.

### SUMMARY OF THE INVENTION

The spine/spinal cord injury varies in symptom depending on the site and the level of the injury, and causes complete or incomplete loss of the motor function or the sensory function, and drug therapy or rehabilitation may improve spasm, the functions of extremities and the like. From the documents (X. Chen et al., Cell Physiol. Biochem. 2018; 47: 176-190 and C. Chen et al., Neurochem. Res. 2010; 35(7): 1111-1118), it is known that in spinal cord injury models, molecular hydrogen protects nerve cells of the spinal cord, but use of molecular hydrogen for improving symptoms of complications of spine/spinal cord injury, which require long-term care, for example spasm (also referred to as spasticity), pain, autonomic nervous system dysfunction and burning sensation, is not known.

An object of the present invention is to provide a composition for improving a symptom (e.g. aftereffects) of spine/spinal cord injury.

Specifically, the present invention includes the following characteristics.

(1) A composition for improving a symptom of spine/spinal cord injury in a subject, the composition comprising molecular hydrogen as an active ingredient.

(2) The composition according to (1), wherein the spine/spinal cord injury is selected from the group consisting of cervical spine injury and/or cervical cord injury, thoracic spine injury and/or thoracic cord injury, lumbar spine injury and/or lumbar cord injury, sacral spine injury and/or sacral cord injury, and hernia.

(3) The composition according to (1) or (2), wherein the symptom is selected from the group consisting of pain, disordered sleep, decreased appetite, physical fatigue, mental fatigue, nervous fatigue, burning sensation on the back and hand and foot spasticity which are caused by the spine/spinal cord injury.

(4) The composition according to any of (1) to (3), wherein the composition is a liquid or a gas containing the molecular hydrogen.

(5) The composition according to (4), wherein the liquid containing the molecular hydrogen has a hydrogen concentration of 1 to 10 ppm.

(6) The composition according to (4), wherein the gas containing the molecular hydrogen has a hydrogen concentration of 0.5 to 18.5 vol %.

(7) The composition according to any of (1) to (6), wherein the subject is a mammal including human and dog.

(8) The composition according to any of (1) to (7), wherein the composition is prepared using a hydrogen gas generating apparatus, a hydrogen water generating apparatus or a hydrogen gas adding apparatus.

(9) A method for improving a symptom of spine/spinal cord injury in a subject having spine/spinal cord injury, the method comprising administering to the subject the composition described in any of (1) to (8).

The invention improves (e.g. alleviates or suppresses) a symptom of a patient having spine/spinal cord injury (e.g. pain, disordered sleep, decreased appetite, physical fatigue, mental fatigue, nervous fatigue, burning sensation on the back, hand and foot spasticity or the like which is caused by the spine/spinal cord injury).

### BRIEF DESCRIPTION OF THE DRAWINGS

FIG. 1 shows scores for the described items on Face Pain Scale (FPS; scale: 0 to 5) over a period from Oct. 4, 2018 to Oct. 31, 2018 for a patient in Example described later;

FIG. 2 shows scores for the described items on Face Pain Scale (FPS; scale: 0 to 5) over a period from Nov. 1, 2018 to Nov. 30, 2018 for the patient in Example described later;

FIG. 3 shows scores for the described items on Face Pain Scale (FPS; scale: 0 to 5) over a period from Dec. 1, 2018 to Dec. 31, 2018 for the patient in Example described later;

FIG. 4 shows scores for the described items on Face Pain Scale (FPS; scale: 0 to 5) over a period from Jan. 1, 2019 to Jan. 31, 2019 for the patient in Example described later;

FIG. 5 shows scores for the described items on Face Pain Scale (FPS; scale: 0 to 5) over a period from Feb. 1, 2019 to Feb. 14, 2019 for the patient in Example described later; and

FIG. 6 shows scores for the described items on Face Pain Scale (FPS; scale: 0 to 5) on and after May 13, 2019 for the patient in Example described later.

### DETAILED DESCRIPTION

The present invention will be described in more detail.

### 1. Composition for Improving Symptom of Spine/Spinal Cord Injury

The present invention provides a composition for improving a symptom of spine/spinal cord injury in a subject, the composition comprising molecular hydrogen as an active ingredient.

In this specification, the “spine/spinal cord injury” is selected from the group consisting of cervical spine/cervical cord injury, thoracic spine/thoracic cord injury, lumbar spine/lumbar cord injury, sacral spine/sacral cord injury and hernia.

The spine/spinal cord injury includes traumatic spine/spinal cord injury caused by, for example, a traffic accident, a downfall, a fall, a sports accident, an accident of collision with a falling object or an accident of being crushed by a collapsed object, and a non-traumatic spine/spinal cord injury such as disease (e.g. spinal cord tumor) or malformation (e.g. spinal artery malformation).

The human spine includes seven cervical spines, twelve thoracic spines, five lumbar spines and a sacral spine, and the spinal cord is protected by the spine. Here, the spinal cord includes a cervical cord, thoracic cord, a lumbar cord and a sacral cord.

The spine/spinal cord injury is spine injury and/or spinal cord injury, and is called, for example, cervical spine injury and/or cervical cord injury, thoracic spine injury and/or thoracic cord injury, lumbar spine injury and/or lumbar cord injury or sacral spine injury and/or sacral cord injury, depending on the site of injury in the spine and the spinal cord. Damage of the spine and/or impairment of the spinal cord cause spine injury and/or spinal cord injury.

The hernia is a disease in which the cartilage (intervertebral disc) serving as a cushion between vertebras of the lumbar part of the backbone degenerates, so that some of tissues protrude. The hernia is sometimes referred to as intervertebral disc hernia.

In this specification, examples of the “symptom of spine/spinal cord injury” include pain, disordered sleep, decreased appetite, physical fatigue, mental fatigue, nervous fatigue, burning sensation on the back, hand and foot spasticity and paralysis which are caused by the spine/spinal cord injury.

The physical fatigue, the mental fatigue and the nervous fatigue generally refer to fatigue of the body, fatigue of the mind and fatigue of the brain, respectively. These three types of fatigue are interrelated, and when the fatigue becomes chronic, disorder of the autonomic nervous system may occur, leading to adverse effects on sleep, appetite, spirit and the like.

The hand and foot spasticity (spasm) refers to a state in which the muscle is so tense that it is difficult to move hands and feet, the neck and the back warp, or spontaneous movement occurs. The spasticity is associated with a symptom in which, for example, a hand is kept fisted and hardly opened although an attempt is made to open the fisted hand, an elbow bends, or toes bend toward the back side of the foot.

The burning sensation on the back refers to an unusual sensation of feeling burning-hot at an area ranging from the back to the waist, and this may cause burning pain.

The pain is said to be experienced by about 70% or more of the patients after spine/spinal cord injury. About 40% of the patients feel pain at all times, and resultantly suffer from disordered sleep. In such patients, the pain is also caused by a climate change, burning pain, numb pain, autonomic hyperreflexia or the like.

The paralysis includes motor paralysis and anesthesia.

The term “subject” as used herein includes mammals, for example primates including humans, pet animals such as dogs and cats, and ornamental animals such as those in zoos. Preferred subjects are humans and dogs.

In this specification, the “hydrogen” which is an active ingredient of the composition of the present invention is molecular hydrogen (i.e. gaseous hydrogen or hydrogen gas), and is sometimes referred to simply as “hydrogen” or “hydrogen gas” unless otherwise specified. The term “hydrogen” as used herein refers to hydrogen having a molecular formula of H₂, D₂ (deuterium) or HD (hydrogen deuteride), or a mixed gas thereof. D₂ is expensive, and is known to have a superoxide eliminating action higher than that of H₂. The hydrogen usable in the present invention is H₂, D₂ (deuterium), HD (hydrogen deuteride) or a mixed gas thereof, preferably H₂. Alternatively, D₂ and/or HD may be used instead of H₂ or in combination with H₂.

The preferred form of the composition of the present invention is a molecular hydrogen-containing gas or liquid.

The molecular hydrogen-containing gas is preferably air containing hydrogen gas, or a mixed gas containing hydrogen gas and oxygen gas. The hydrogen gas concentration of the molecular hydrogen-containing gas is more than zero (0) and not more than 18.5 vol %, for example 0.5 to 18.5 vol %, preferably 1 to 10 vol %, for example 2 to 10 vol %, 2 to 9 vol %, 2 to 8 vol %, 3 to 10 vol %, 3 to 9 vol %, 3 to 8 vol %, 3 to 7 vol %, 3 to 6 vol %, 4 to 10 vol %, 4 to 9 vol %, 4 to 8 vol %, 4 to 7 vol %, 4 to 6 vol %, 4 to 5 vol %, 5 to 10 vol %, 5 to 9 vol %, 5 to 8 vol %, 6 to 10 vol %, 6 to 9 vol %, 6 to 8 vol % or 6 to 7 vol %. In the present invention, the improving (e.g. suppressing or alleviating) effect on a symptom caused by spine/spinal cord injury tends to be enhanced as the hydrogen gas concentration increases within the bounds of not exceeding the explosion limit or as the daily dosage of hydrogen increases.

Since hydrogen is a combustible or explosive gas, it is preferable that in improvement of a symptom caused by spine/spinal cord injury, hydrogen incorporated in the composition of the present invention under conditions ensuring safety of a subject such as a human be administered to the subject.

When the gas other than hydrogen gas is air, the concentration of the air is in the range of, for example, 81.5 to 99.5 vol %.

When the gas other than hydrogen gas is a gas containing oxygen gas, the concentration of the oxygen gas is in the range of, for example, 21 to 99.5 vol %.

The composition may contain, for example, nitrogen gas as another main gas.

The molecular hydrogen-containing liquid is specifically an aqueous liquid in which hydrogen gas is dissolved. Here, examples of the aqueous liquid include, but are not limited to, water (e.g. purified water and sterilized water), physiological saline solutions, buffer solutions (e.g. buffer solutions having a pH of 4 to 7.4), drip-feed solutions, infusion solutions, injection solutions, and beverages (e.g. tea beverages such as green tea and black tea, fruit juices, green juices and vegetable juices). The hydrogen concentration of the molecular hydrogen-containing liquid is not limited, and is, for example 1 to 10 ppm, preferably 1.2 to 9 ppm, for example 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm or 5 to 7 ppm. In the present invention, the improving (e.g. suppressing or alleviating) effect on a symptom caused by spine/spinal cord injury tends to be enhanced as the dissolved hydrogen concentration increases within the bounds of not exceeding the explosion limit or as the daily dosage of hydrogen increases.

A pharmaceutical product for treating a symptom of spine/spinal cord injury may be added to the molecular hydrogen-containing liquid. Alternatively, the pharmaceutical product may be administered separately from the molecular hydrogen-containing liquid or gas. Examples of the pharmaceutical product include, but are not limited to, analgesic drugs (e.g. Voltaren, Loxonin and morphine), and antispasmogenic drugs (e.g. baclofen and tizanidine).

The molecular hydrogen-containing gas or liquid is blended so as to give a predetermined hydrogen gas concentration, and then filled into, for example, a pressure-resistant container (e.g. stainless cylinder, an aluminum can, a pressure-resistant plastic bottle (e.g. a pressure-resistant PET bottle) or a plastic bag preferably laminated on the inside with an aluminum film, or an aluminum bag). Aluminum has a property of being hardly permeable to hydrogen molecules. Alternatively, at the time of administration, the molecular hydrogen-containing gas or the molecular hydrogen-containing liquid may be prepared on site by using an apparatus such as a hydrogen gas generating apparatus, a hydrogen water generating apparatus or a hydrogen gas adding apparatus, for example a known or commercially available hydrogen supplying apparatus (apparatus for generating the molecular hydrogen-containing gas), hydrogen adding device (apparatus for generating hydrogen water) or non-destructive hydrogen injecting device (apparatus for non-destructively adding hydrogen gas into, for example, a biocompatible liquid bag such as a drip-feed solution).

By the hydrogen gas supplying apparatus, hydrogen gas produced from reaction of a hydrogen generator (e.g. metallic aluminum or hydrogenated magnesium) with water can be mixed with a diluting gas (e.g. air or oxygen) at a predetermined ratio (e.g. Japanese Patent No. 5228142). Alternatively, hydrogen gas produced through electrolysis of water is mixed with a diluting gas such as oxygen or air (e.g. Japanese Patent No. 5502973 and Japanese Patent No. 5900688). This enables preparation of a molecular hydrogen-containing gas having a hydrogen concentration within the range of, for example, 0.5 to 18.5 vol %.

The hydrogen adding device is an apparatus which produces hydrogen using a hydrogen generator and a pH adjuster, and dissolves the hydrogen in a biocompatible liquid such as water (e.g. Japanese Patent No. 4756102, Japanese Patent No. 4652479, Japanese Patent No. 4950352, Japanese Patent No. 6159462, Japanese Patent No. 6170605 and Japanese Patent Laid-Open No. 2017-104842). The combination of a hydrogen generator and a pH adjuster is, for example, a combination of metallic magnesium and an ion-exchange resin or an organic acid (e.g. malic acid or citric acid), or a combination of metallic aluminum powder and calcium hydroxide powder. This enables preparation of a molecular hydrogen-containing liquid having a dissolved hydrogen concentration of, for example, about 1 to 10 ppm.

The non-destructive hydrogen injecting device is an apparatus or a device which adds hydrogen gas to a commercially available biocompatible liquid such as a drip-feed solution (encapsulated in, for example, a hydrogen-permeable plastic bag such as a polyethylene bag) from outside the package thereof. The non-destructive hydrogen injecting device is commercially available from, for example, MiZ Company Limited (Kanagawa, Japan) (http://www.e-miz.co.jp/technology.html). This apparatus is capable of aseptically dissolving hydrogen in a biocompatible liquid until achievement of equilibrium by immersing a bag containing the biocompatible liquid in saturated hydrogen water to make hydrogen pass into the bag. The apparatus includes, for example, an electrolytic bath and a water bath, where water in the water bath can be electrolyzed to produce hydrogen while circulating through the electrolytic bath and the water bath. Alternatively, a simplified disposable device can be used for the same purpose (e.g. Japanese Patent Laid-Open No. 2016-112562). The device includes a biocompatible liquid-containing plastic bag (hydrogen-permeable bag, for example a polyethylene bag) and a hydrogen generator (e.g. metallic calcium or metallic magnesium/cation-exchange resin) in an aluminum bag, and the hydrogen generator is wrapped in, for example, a nonwoven fabric (e.g. water vapor-permeable nonwoven fabric). Hydrogen produced by wetting the hydrogen generator wrapped in the nonwoven fabric with a small amount of water such as water vapor is non-destructively and aseptically dissolved in the biocompatible liquid.

Alternatively, a molecular hydrogen-containing gas or liquid adjusted to a predetermined hydrogen concentration and a predetermined oxygen or air concentration may be prepared using a purified hydrogen gas cylinder, and a purified oxygen gas cylinder or a purified air cylinder.

The molecular hydrogen-containing gas or the molecular hydrogen-containing liquid (e.g. water (e.g. purified water or sterilized water), physiological saline solution or drip-feed solution) prepared using the above-described apparatus or device can be orally or parenterally administered to a subject having spine/spinal cord injury.

Another form of the composition of the present invention includes a dosage form which is configured to be orally administered to (or ingested into) a subject and which contains a hydrogen generator enabling production of hydrogen in the digestive tract (e.g. tablet or capsule). Preferably, the hydrogen generator is made up of ingredients approved as, for example, food or food additives.

The method for administering the composition of the present invention to a subject is preferably pulmonary administration by, for example, inhalation or aspiration when molecular hydrogen is an active ingredient. Oral administration or intravenous administration (including drip infusion) is preferable when a molecular hydrogen-containing liquid is an active ingredient. When gas is inhaled, the gas can be inhaled from the mouth or the nose through a nasal cannula or a mask-type device covering the mouth and the nose, sent to the lung, and delivered to every part of the body through blood.

The molecular hydrogen-containing liquid to be orally administered may be cooled or stored at normal temperature, and administered to a subject. It is known that hydrogen is dissolved in water at a concentration of about 1.6 ppm (1.6 mg/L) at normal temperature and normal pressure, and has relatively small variability in solubility depending on the temperature. Alternatively, the molecular hydrogen-containing liquid may be administered to a subject through a route of parenteral administration such as intravenous administration or intraarterial administration when the molecular hydrogen-containing liquid is in the form of a hydrogen gas-containing drip-feed solution or injection solution prepared using, for example, the above-described non-destructive hydrogen injecting device.

The molecular hydrogen-containing gas having the above-described hydrogen concentration or the molecular hydrogen-containing liquid having the above-described dissolved hydrogen concentration can be administered to a human once or a plurality of times (e.g. 2 or 3 times) a day over the period of 1 week to 3 months or more, for example 1 week to 6 months or more (e.g. 1 year or more or 2 years or more). When the molecular hydrogen-containing gas is administered, it is preferable to inhale the gas for at least 30 minutes per inhalation. Since the improving effect is enhanced as the inhalation time increases, the gas can be administered for 30 minutes to 1 hour, 1 hour to 2 hours, 2 hours to 3 hours or more, for example, per halation. When the molecular hydrogen-containing gas is administered through pulmonary administration by inhalation or aspiration, the gas can be administered to a subject in an environment at atmospheric pressure, or in an environment (including the molecular hydrogen-containing gas) at a high pressure within the range of, for example, more than standard atmospheric pressure (i.e. about 1.013 atm) and not more than 7.0 atm, for example an environment at a high pressure within the range of 1.02 to 7.0 atm, preferably 1.02 to 5.0 atm, more preferably 1.02 to 4.0 atm, still more preferably 1.02 to 1.35 atm.

### 2. Method for Improving Symptom of Spine/Spinal Cord Injury

Further, the present invention provides a method for improving a symptom of spine/spinal cord injury in a subject having spine/spinal cord injury, the method comprising administering the composition to the subject.

The composition containing molecular hydrogen, the spine/spinal cord injury and symptoms thereof, the dosage, the administration method and the like are as described in the above section 1.

The method of the present invention allows the composition of the present invention to be administered to a subject having a symptom of spine/spinal cord injury in the earliest possible stage after spine/spinal cord injury (e.g. one to seven days after spine/spinal cord injury), and the method of the present invention is also effective even if the composition is administered, for example, 10 days to 1 month or more (e.g. 6 months or more, 1 year or more, 2 years or more, 3 years or more, or 6 years or more) after the onset.

In the method of the present invention, a gas (preferably air or oxygen) containing molecular hydrogen in an amount of, for example, 0.5 to 18.5 vol % (e.g. 2 to 10 vol %, 2 to 9 vol %, 2 to 8 vol %, 3 to 10 vol %, 3 to 9 vol %, 3 to 8 vol %, 3 to 7 vol %, 3 to 6 vol %, 4 to 10 vol %, 4 to 9 vol %, 4 to 8 vol %, 4 to 7 vol %, 4 to 6 vol %, 4 to 5 vol %, 5 to 10 vol %, 5 to 9 vol %, 5 to 8 vol %, 6 to 10 vol %, 6 to 9 vol %, 6 to 8 vol % or 6 to 7 vol %, preferably 5 to 10 vol % or 5 to 8 vol %, for example 6 to 10 vol %, 6 to 8 vol % or 6 to 7 vol %) can be inhaled or aspirated into a subject having a symptom of spine/spinal cord injury for 1 to 3 hours or more, for example, a day, over the period of, for example, 1 to 3 months or more, 4 to 7 months or more, or 1 to 3 years or more.

Alternatively, in the method of the present invention, a liquid containing molecular hydrogen in an amount of, for example, 1 to 10 ppm (e.g. 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm or 5 to 7 ppm, preferably 3 to 10 ppm, 4 to 10 ppm, 5 to 10 ppm, 5 to 9 ppm, 5 to 8 ppm or 5 to 7 ppm) can be administered to a subject having a symptom of spine/spinal cord injury at a dose of, for example, 200 to 500 mL per administration in the case of intravenous administration and at a dose of, for example, 500 to 1000 mL in the case of oral administration, over the period of, for example, 0.5 to 3 months or more, 4 to 7 months or more, or 1 to 3 years or more.

In any of the administration forms, it is possible to determine an administration period while observing the degree of improvement of a symptom of spine/spinal cord injury. For example, the administration may be continued if a sign of improvement of the symptom of spine/spinal cord injury is produced, and the administration may be stopped if remission is obtained.

The method of the present invention can be used in combination with treatment or rehabilitation for a symptom of spine/spinal cord injury (e.g. pain, disordered sleep, decreased appetite, physical fatigue, mental fatigue, nervous fatigue, burning sensation on the back, hand and foot spasticity (also referred to as spasm) or paralysis). Use of the method in combination with the treatment or rehabilitation is expected to enhance the improving effect on a symptom of spine/spinal cord injury.

As the rehabilitation, physical therapy, technical training activity and counseling for meeting social and emotional needs can be appropriately combined.

With regard to the treatment, the method may be used in combination with drug therapy for a symptom such as pain or spasticity. Examples of the drug include analgesic drugs (e.g. Voltaren, Loxonin and morphine), and antispasmogenic drugs (e.g. baclofen and tizanidine).

### EXAMPLES

The present invention will be described in more detail by way of Example below, which should not be construed as limiting the present invention.

### Example 1

### <Example of Improvement of Symptom in Cervical Spine/Cervical Cord Injury by Inhalation of Hydrogen>

A patient (male, 36 years old) had an accident causing cervical spine/cervical cord injury when he bumped his head against the sea bed in diving into shallow water. He restored consciousness about a week after the accident. He had sensation in the body part under the neck (site of injury), but was unable to move the part on his will. Thereafter, during about 6 years, he underwent an operation for the site of injury and rehabilitation to slowly recover, but there was a limit on improvement of the symptom. Specifically, the part under the biceps muscle of arm was incompletely paralyzed, an unusual sensation of feeling burning-hot (burning sensation) at an area ranging from the back to the waist lasted, and spasticity persisted on both feet. He tended to suffer from disordered autonomic nerves, was unable to get sufficient sleep, was not mentally relaxed because of the handicapped body, and often delivered associated complaints to people around him.

Immediately before inhalation of hydrogen gas 6 years after the accident, he went to work in the afternoon 4 or 5 days a week, and stayed at home throughout a day when he felt ill. Further, he occasionally saw a doctor of psychiatry.

About 6 years after the accident, the patient underwent inhalation of hydrogen (hydrogen inhalation equipment: MHG-2000a Type, hydrogen concentration: 6 to 7.5%, hydrogen gas production rate: 140 mL/min, MiZ Company Limited (Kanagawa, Japan) over a period from Oct. 4, 2018 to May 12, 2019. Hydrogen was aspirated twice a day, once in the morning and once in the afternoon, for a total of 2 to 7.5 hours (an average of 3.3 hours a day), and as symptoms, pain, disordered sleep, decreased appetite, physical fatigue, mental fatigue, nervous fatigue, burning sensation on the back and foot spasticity were evaluated on Face Pain Scale (FPS; scale: 0 to 5) for about 7 months. For each of the symptoms, FPS “0” indicates that the physical state is very good, FPS “1” indicates that the physical state is good, FPS “2” indicates that there is no change, FPS “3” indicates that the physical state is slightly bad, FPS “4” indicates that the physical state is bad, and FPS “5” indicates that the physical state is very bad.

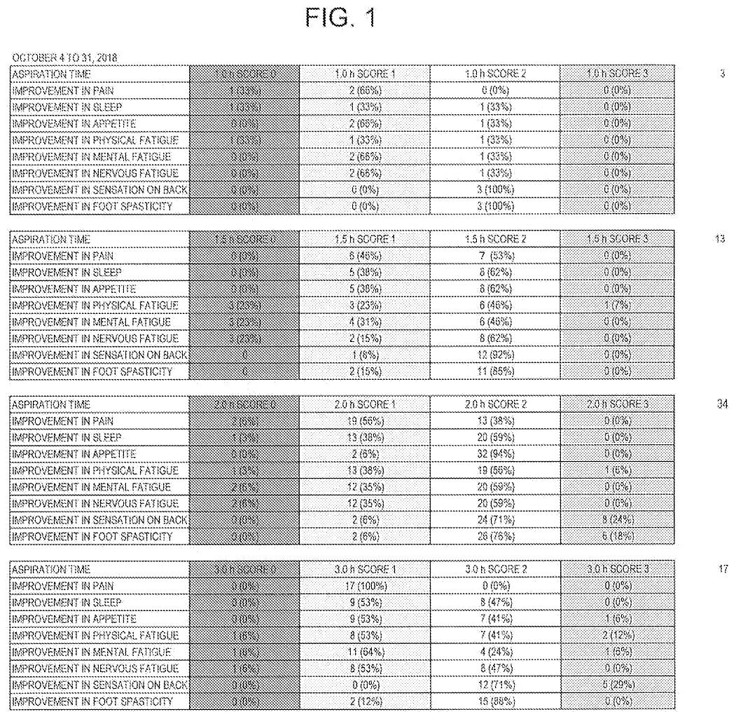

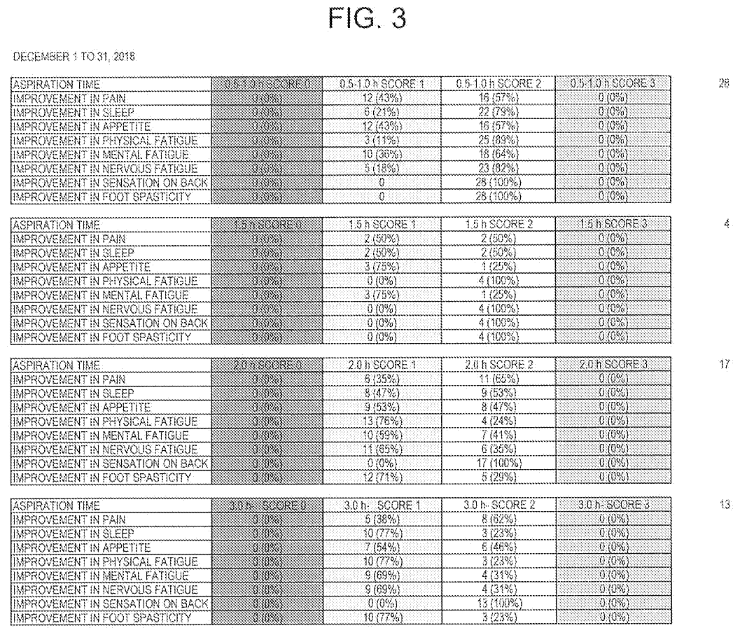

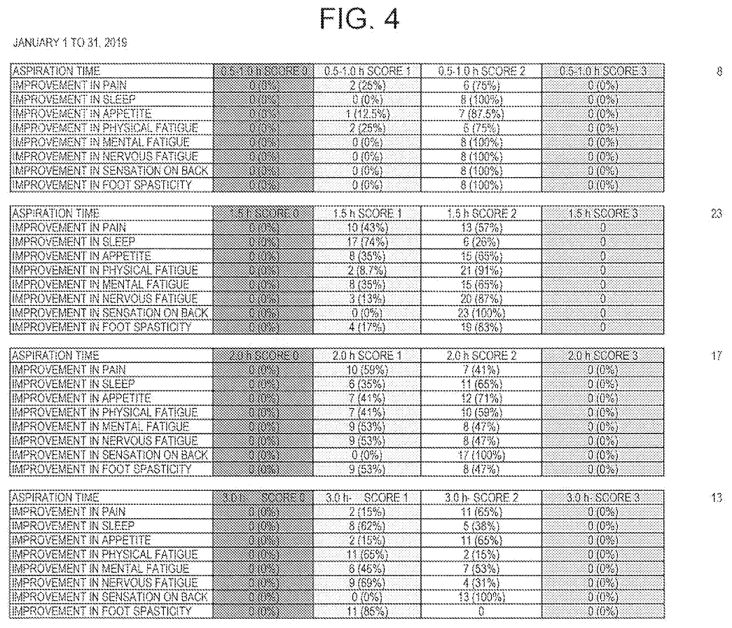

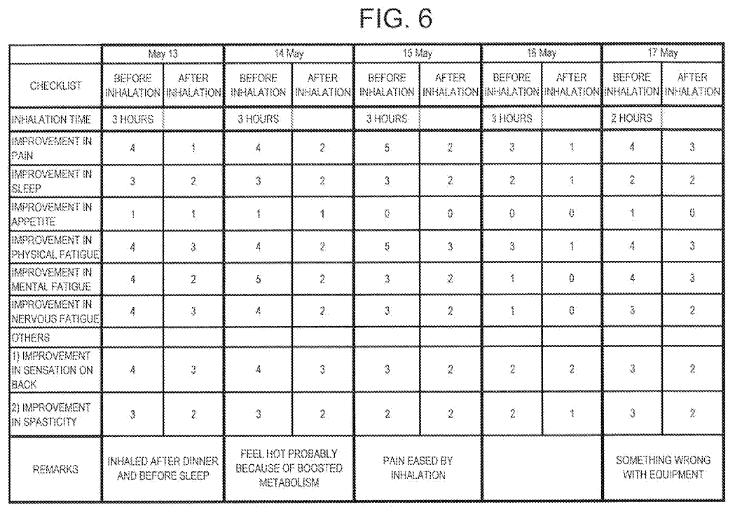

The FAS scales in FIGS. 1 to 5 indicate how the state changed after inhalation as compared to the state before inhalation. The FAS scale in FIG. 6 shows a difference between the sensation before inhalation and the sensation after inhalation. In FIGS. 1 to 5, the number of scores (e.g. 0 to 3) was counted for each of the periods, and the ratio (%) of the scores in each item was calculated. In the diagrams, the numbers shown outside the table represent the total numbers of respective scores.

Further, on May 13, 2019, the patient started inhalation of hydrogen using hydrogen gas inhalation equipment for professional use (hydrogen concentration: 6 to 7.5%, hydrogen gas production rate: 1,120 mL/min, MiZ Company Limited) instead of the hydrogen inhalation equipment MHG-2000a Type.

FIGS. 1 to 5 show the results of counting over a period from Oct. 4, 2018 to Feb. 14, 2019. For the counting, monthly data was prepared in the following manner: the number of FAS “1” to “5” scores for each item was counted and summed for each inhalation time (FIGS. 1 to 5; note that FAS “4” and FAS “5” scores were not recorded at all, and therefore these scores are not given in FIGS. 1 to 5).

The results of counting over a period from Oct. 4, 2018, i.e. the day of starting inhalation of hydrogen gas, to Oct. 31, 2018 show that there was a good improvement tendency in the items of “improvement in pain”, “improvement in sleep”, “improvement in appetite”, “improvement in physical fatigue”, “improvement in mental fatigue” and “improvement in nervous fatigue” because FAS “0” and FAS “1” scores were recorded after the start of inhalation of hydrogen gas. However, for the items of “improvement in sensation on the back” and “improvement in foot spasticity”, the improvement tendency was unknown or became worse because FAS “2” and FAS “3” scores were recorded although FAS “1” scores were occasionally recorded. This is because the aftereffects of cervical spine/cervical cord injury exceeded the effect of improvement by inhalation because evaluation was performed immediately after the start of inhalation. It is apparent that continuous inhalation of hydrogen gas improved the aftereffects of spine/cervical cord injury because FAS “3” or greater scores were not recorded in any of the items on and after Dec. 1, 2018 ([FIG. 1]).

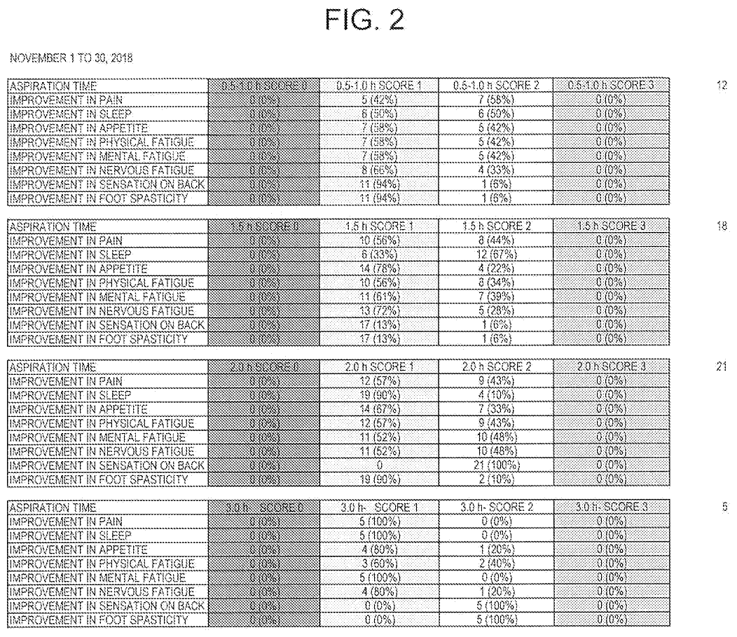

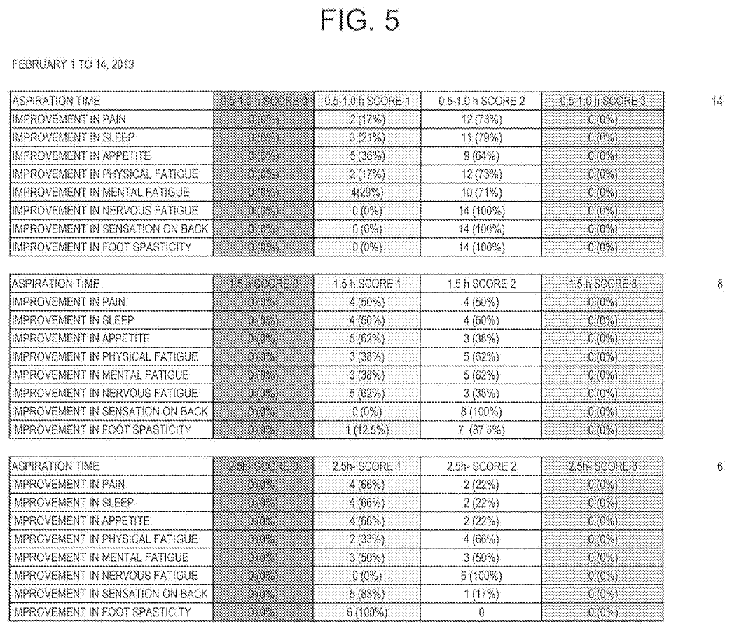

In and after November 2018, the ratio of FAS “1” scores tended to increase as the inhalation time increased, and therefore the improving effect on aftereffects was enhanced as the hydrogen gas inhalation time increased ([FIG. 2] to [FIG. 5]).

Further, on May 13, 2019, the patient started inhalation of hydrogen gas using hydrogen gas inhalation equipment for professional use with a hydrogen gas rate 8 times higher as compared to the hydrogen inhalation equipment MHG-2000a Type. As a result, improvement occurred in all the items ([FIG. 6]).

The patient recorded the FAS scores as well as his feeling before and after inhalation on paper record forms as will be described below.

In the record on Oct. 9, 2018, the patient reported “An improving effect is produced successfully”.

In the record on Oct. 12, 2018, the patient reported “The time which I can spend in peace considerably increases probably because I can secure the time when the parasympathetic nerve dominates, so I think that negative thinking such as irritation, anxiousness, jealousness and envy has weakened. The inhalation performed before sleeping is recommendable. In fact, I can remain calm at the time when the sympathetic nerve dominates, for example during working, and I think that I have been more inclined to make others happy. For the body, I have the feeling that the joints and the muscles have become smoother, and the physical therapist said that mobility and stiffness had improved. Peripheral nerve sensation and transmission are slightly better than they were a week ago.”

In the record on Oct. 15, 2018, the patient reported “I was able to exercise very well when taking a knee bend exercise in a rehabilitation hospital I visit once a week, and I found that I was less often tired and more easily moved the body than before the start of inhalation of hydrogen.”

In the record on Oct. 16, 2018, the patient reported “For the sensation on the back, I aspirate much hydrogen before sleeping, and thus at the time of getting up, the sensation of feeling hot is less intense than before, so that I feel comfortable more often than not. I have found that the spasticity of feet was alleviated to ease movements, resulting in facilitation of rehabilitation. In past days, breathing was disrupted or I felt tired throughout the body, but I feel that the frequency of such cases has decreased since I started inhalation of hydrogen.”

In the record on Nov. 19, 2018, the patient reported “It has been found from today's rehabilitation that I became good at squatting with spasticity of feet because inhalation of hydrogen softened the spasticity of feet to easily perform control. For the unusual sensation on the back, inhalation of hydrogen on the bed tends to lead to a comfortable feeling.”

In the record on Jan. 22, 2019, the patient reported “I have a feeling that the spasticity becomes softer after inhalation of hydrogen. The state is not worsened as I take rehabilitation once a week.” In the record on May 13, 2019, the patient reported “a more potent effect is exhibited in alleviation of spasticity, feeling of reaching the peripheral nerve, boosted metabolism, flow of blood, induction of sleep and the like than before. Metabolism is so intensely boosted that the body temperature rises, and thus sweating cannot be stopped.” In the above report, the hydrogen gas inhalation equipment used by the patient is hydrogen gas inhalation equipment for business use (hydrogen concentration: 6 to 7.5%, hydrogen gas production amount: 1,120 mL/min, MiZ Company Limited).

On Mar. 1, 2019, the patient started intake of high-concentration hydrogen water (“10Water” manufactured by TRUST NETWORK Co., Ltd., hydrogen concentration: 10 ppm, 500 ml), and as a result, he felt the effect of recovery from exhaustion, and realized improvement of blood circulation.

### INDUSTRIAL APPLICABILITY

According to the present invention, symptoms of spine/spinal cord injury (e.g. pain, disordered sleep, decreased appetite, physical fatigue, mental fatigue, nervous fatigue, burning sensation on the back and foot spasticity) can be improved by administrating a composition containing molecular hydrogen.

1. A method for improving a symptom of spine/spinal cord injury in a subject, the method comprising administering to a subject in need thereof molecular hydrogen as an active ingredient.

2. The method according to claim 1,
wherein the spine/spinal cord injury is selected from the group consisting of cervical spine injury and/or cervical cord injury, thoracic spine injury and/or thoracic cord injury, lumbar spine injury and/or lumbar cord injury, sacral spine injury and/or sacral cord injury, and hernia.

3. The method according to claim 1, wherein the symptom is selected from the group consisting of pain, disordered sleep, decreased appetite, physical fatigue, mental fatigue, nervous fatigue, burning sensation on the back and hand and foot spasticity which are caused by the spine/spinal cord injury.

4. The method according to claim 1, wherein the composition is a liquid or a gas containing the molecular hydrogen.

5. The method according to claim 4, wherein the liquid containing the molecular hydrogen has a hydrogen concentration of 1 to 10 ppm.

6. The method according to claim 4, wherein the gas containing the molecular hydrogen has a hydrogen concentration of 0.5 to 18.5 vol %

7. The method according to claim 1, wherein the subject is a mammal.

8. The method according to claim 1, wherein the composition is prepared using a hydrogen gas generating apparatus, a hydrogen water generating apparatus or a hydrogen gas adding apparatus.

9. (canceled)

10. The method of claim 7, wherein the subject is a human or dog.

11. The method according to claim 2, wherein the composition is a liquid or a gas containing the molecular hydrogen.

12. The method according to claim 11, wherein the liquid containing the molecular hydrogen has a hydrogen concentration of 1 to 10 ppm.

13. The method according to claim 11, wherein the gas containing the molecular hydrogen has a hydrogen concentration of 0.5 to 18.5 vol %.

14. The method according to claim 3, wherein the composition is a liquid or a gas containing the molecular hydrogen.

15. The method according to claim 14, wherein the liquid containing the molecular hydrogen has a hydrogen concentration of 1 to 10 ppm.

16. The method according to claim 14, wherein the gas containing the molecular hydrogen has a hydrogen concentration of 0.5 to 18.5 vol %.

17. The method according to claim 2, wherein the symptom is selected from the group consisting of pain, disordered sleep, decreased appetite, physical fatigue, mental fatigue, nervous fatigue, burning sensation on the back and hand and foot spasticity which are caused by the spine/spinal cord injury.

18. The method according to claim 17, wherein the composition is a liquid or a gas containing the molecular hydrogen.

19. The method according to claim 18, wherein the liquid containing the molecular hydrogen has a hydrogen concentration of 1 to 10 ppm.

20. The method according to claim 18, wherein the gas containing the molecular hydrogen has a hydrogen concentration of 0.5 to 18.5 vol %.
